(19) United States
(12) Patent Application Publication
(10) Pub. No.: US 2010/0093984 A1
(43) Pub. Date: Apr. 15, 2010

(54) EXTRACTION METHOD

(76) Inventors: Corinna WIRTH, Heidelberg (DE); Herwig Buchholz, Frankfurt (DE); Alexandre Andrade De Souza Costa, Rio de Janeiro (BR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/637,152

(22) Filed: Dec. 14, 2009

Related U.S. Application Data
(63) Continuation of application No. 11/909,348, filed on Sep. 21, 2007.

(30) Foreign Application Priority Data
Mar. 23, 2005 (DE) 10 2005 013 380.0
Feb. 27, 2006 (EP) PCT/EP2006/001799

(51) Int. Cl. C07H 17/04

(52) U.S. Cl. 536/8

(57) ABSTRACT

The present invention relates to a process for the preparation of tiliroside or tiliroside-containing extracts from parts of a plant selected from the Sterculiaceae family, to corresponding extracts, and to the use of the extracts or process products in cosmetics or topical applications.

The present invention relates to a process for the preparation of tiliroside or tiliroside-containing extracts, to corresponding extracts, and to the use of the extracts or process products in cosmetics or topical applications.

Applications in traditional medicine are known for a very wide variety of plants. Thus, in traditional medicine, roots of Waltheria ovate (lucraco) are boiled with sugar in water by the rural population of Peru and the liquid is drunk as a healthy drink with food. In addition, it has been used against bladder inflammation and other inflammatory diseases. No traditional application of the related species Waltheria paniculata is known. Waltheria douradinha is used in Brazil against bronchitis and for cleaning badly healing wounds (Morel, Ademir et al, A new cyclopeptide alkaloid from the bark of Waltheria douradinha, Tetrahedron letters, 40, (1999) 9205-9209).

WO 02/69926 describes various flavone derivatives and in particular tiliroside (kaempferol-3-(6″-p-coumarylglucoside)). The suitability of these compounds for use as UV filters and the use as active ingredient for protection against oxidative stress and for the prevention of skin ageing are described. It is furthermore described that these compounds exhibit antiallergic, antiinflammatory, inflammation-inhibiting and antiirritative properties and can thus be used for the treatment or preventative treatment of allergies, inflammation and irritations, in particular of the skin.

DE 195 44 905 A1 describes, for example, a process for the preparation of plant extracts comprising tiliroside and the use of the plant extracts in medicaments and food products.

DE 199 22 287 A1 describes tiliroside as a starting flavonoid for the preparation of tiliroside esters whose acid unit contains 3 to 30 C atoms. These esters are used in cosmetics. However, DE 199 22 287 A1 does not describe compositions comprising tiliroside.

The European patent application with the application number EP 03015616.0 describes the suitability of tiliroside for the treatment of eczema. The compounds are particularly advantageous here in the treatment of atopic eczema, such as, in particular, milk crust, neurodermatitis, prurigo and dermatitis sicca. The compounds can greatly reduce the acute symptoms, reduce the frequency of occurrence of acute symptoms and generally contribute to improving the skin picture.

WO 02/69926 states that tiliroside can be obtained, for example, from the plants of the genera Althaea, Aristolochia, Helianthemum, Lindera, Magnolia, Platanus, Potentilla, Quercus, Rosa, Sida, Sorbus and/or Tilia, where the following species are preferred: Althaea officinalis, Althaea rosea, Aristolochia heterophylla, Helianthemum glomeratum, Lindera megaphylla, Magnolia salicifolia, Platanus acerifolia, Platanus occidentalis, Potentilla anserina, Quercus pubescens, Quercus sober, Quercus laurifolia, Quercus ilex, Quercus imbricaria, Quercus virginiana, Rosa pomifera, Sida rhombifolia, Sida poeppigiana, Sida cordifolia, Sida glaziovii, Sorbus pendula, Tilia argenta and Tilia cordata. According to WO 02/69926, tiliroside is particularly preferably obtained from the plant Sida glaziovii.

Surprisingly, it has now been found that tiliroside can particularly advantageously be obtained from the Sterculiaceae family.

The present invention therefore relates firstly to a process for the preparation of tiliroside from plant material, characterised in that parts of a plant selected from the Sterculiaceae family are extracted, and the crude extract is processed further.

It has been found here that tiliroside is readily accessible, in particular, from Waltheria species, where the Waltheria species is preferably Waltheria americana, Waltheria douradinha, Waltheria paniculata, Waltheria indica, Waltheria viscosissima, Waltheria antennas, Waltheria ovata, Waltheria tomentosa, Waltheria madagascariensis, Waltheria glomerata, Waltheria bicolor, Waltheria fryxellii, Waltheria lundelliana, Waltheria tridentata, Waltheria operculata, Waltheria bracteosa, Waltheria macropoda, Waltheria caroliniana, Waltheria arenicola, Waltheria melochia, Waltheria acuminata, Waltheria theobroma, Waltheria indivia or Waltheria taiwana and particularly preferably Waltheria paniculata.

Any desired plant parts can be employed in the process according to the invention. The process can be carried out, for example, with the entire plant or plants. Advantageously, however, the above-ground parts of these plants, for example stems, leaves, flowers and/or buds, are employed, the process having proven particularly economical if the plant parts are leaves.

After collection, the plants or plant parts in question are, if necessary, dried and comminuted and then extracted, where the extractant used can advantageously be selected from the group of water, aqueous solutions of various pH, C₁-C₆-alcohols, ketones (acetone, methyl ketone, diethyl ketone), halogenated hydrocarbons, esters (ethyl acetate, propyl acetate, butyl acetate or analogues), monoalcohols or polyols (glycols, diethylene glycol, propanediol, dipropylene glycol, butylene glycol) or mixtures of at least two of the said substances.

In a preferred variant of the process according to the invention, the plant parts are comminuted in a first step and extracted with a polar organic solvent, preferably at elevated temperature, in a second step.

The organic solvents employed are preferably alcohols, preferably methanol or ethanol, and the extraction is particularly preferably carried out under reflux.

Crude tiliroside can be obtained from the solution by precipitation or evaporation of the solvent. To this end, water is preferably added to the resultant solution, which is subsequently evaporated and allowed to cool.

For purification, solids which precipitate at a temperature in the range 0° C.-25° C. are, in a preferred process variant, filtered off immediately after the addition of water.

In preferred process variants, the crude tiliroside is subsequently purified, where the purification is in turn preferably carried out by recrystallisation or washing.

A process variant which is particularly preferred in accordance with the invention is characterised in that the plant parts are
a. comminuted in a first step and
b. extracted with a polar organic solvent, preferably at elevated temperature, in a second step,
c. water is added to the solution in a third step,
d. the solution is concentrated in a fourth step, and
e. crude tiliroside is precipitated by cooling in a fifth step.

The present invention furthermore relates to a process for the preparation of a plant extract in which parts of Waltheria paniculata are extracted.

Any desired plant parts can, as described above, also be employed in this process, where the plant parts are preferably leaves.

In a preferred variant of the process for the preparation of a plant extract, the process is characterised in that the plant parts are comminuted in a first step and extracted with a polar organic solvent, preferably at elevated temperature, in a second step. The statements made above regarding the corresponding process steps of tiliroside preparation apply correspondingly to these preferred process steps. Tiliroside-containing extracts can thus be obtained analogously to the above-described process for the preparation of tiliroside by omitting one or more purification steps in the preparation of tiliroside.

Extracts from Waltheria species, in particular Waltheria paniculata, which are a further subject-matter of the present invention, or crude tiliroside can, however, also be obtained in accordance with the invention in other process variants.

Thus, a process for the preparation of an aqueous extract can comprise, for example, the following steps:
suspension of the comminuted plant material in water in a reaction vessel;
extraction at 85-90° C. for one hour with stirring;
cooling to ambient temperature;
centrifugation or coarse filtration;

A process variant which is particularly preferred in accordance with the invention is characterised in that the plant parts are
a. comminuted in a first step and
b. extracted with a polar organic solvent, preferably at elevated temperature, in a second step,
c. water is added to the solution in a third step,
d. the solution is concentrated in a fourth step, and
e. crude tiliroside is precipitated by cooling in a fifth step.

The present invention furthermore relates to a process for the preparation of a plant extract in which parts of Waltheria paniculata are extracted.

Any desired plant parts can, as described above, also be employed in this process, where the plant parts are preferably leaves.

In a preferred variant of the process for the preparation of a plant extract, the process is characterised in that the plant parts are comminuted in a first step and extracted with a polar organic solvent, preferably at elevated temperature, in a second step. The statements made above regarding the corresponding process steps of tiliroside preparation apply correspondingly to these preferred process steps. Tiliroside-containing extracts can thus be obtained analogously to the above-described process for the preparation of tiliroside by omitting one or more purification steps in the preparation of tiliroside.

Extracts from Waltheria species, in particular Waltheria paniculata, which are a further subject-matter of the present invention, or crude tiliroside can, however, also be obtained in accordance with the invention in other process variants.

Thus, a process for the preparation of an aqueous extract can comprise, for example, the following steps:
suspension of the comminuted plant material in water in a reaction vessel;
extraction at 85-90° C. for one hour with stirring;
cooling to ambient temperature;
centrifugation or coarse filtration;
if desired clarification using fine filters;
extraction and treatment of the moist residue under the same conditions in order to obtain a second extract;
dewatering of the two extracts by spraying the plant extract, if desired after addition of an assistant, such as maltodextrin (⅔ assistant, ⅓ extracted material).

A process for the preparation of an aqueous/alcoholic extract can comprise, for example, the following steps:
suspension of the comminuted plant material in aqueous ethanol in a reaction vessel;
extraction under reflux for one hour with stirring;
filtration in a Bühner apparatus provided with a fine filter;
collection of the supernatant, concentration of the ethanol phase, if desired centrifugation and filtration;
dewatering by direct spraying of the plant extract.

A process for the preparation of an alcoholic extract can comprise, for example, the following steps:
suspension of the comminuted plant material in ethanol;
extraction under reflux;
cooling;
filtration;
evaporation of the alcohol;
drying.

It has been found that extracts or pure substances obtained by the process described above have properties and actions against free radicals and/or against ageing and properties and/or actions which stimulate the autosynthesis of reduced glutathione and/or antiinflammatory action on topical application.

The present invention consequently furthermore relates to the use of at least one extract from Waltheria paniculata or a pure substance obtained from a Waltheria species for the preparation of a cosmetic product or as active ingredient for topical use on the skin, the mucous membranes and/or the body appendages.

This action can particularly advantageously be used in cosmetic preparations against ageing, against physical stresses (UV rays), cold, heat, wind and against chemical stresses (in particular environmental pollution), in light-protection compositions and in hair products against stresses and light-protection hair products or also in sun compositions and aftersun products.

The present invention furthermore relates to the corresponding compositions for topical use on the skin, the mucous membranes or the body appendages, characterised in that they comprise, as active ingredient, at least one plant extract from Waltheria paniculata or a pure substance obtained from a Waltheria species.

Preferred compositions comprise between 0.001% by weight and 20% by weight, preferably between 0.1% by weight and 10% by weight and particularly preferably 0.5 to 5% by weight of the plant extract or pure substance.

According to a particular embodiment of the invention, the plant extract consists or the plant extracts consist of a purified isolated fraction extracted from one or more of these plants or of a plurality of purified isolated fractions extracted from one or more of these plants.

On the basis of initial indications, it is furthermore assumed that the compositions have the following advantageous properties:
protease inhibition (in particular collagenase and/or elastase)
UV protection, in particular UV-A and/or UV-B protection,
tyrosinase inhibition
stimulation of cell metabolism
inhibition of protein glycation
antiinflammatory action.

The extracts or pure substances are preferably used in cosmetic compositions for skin and hair treatment. These compositions can be emulsions, wax/fat compositions, stick preparations, powders or ointments. Waltheria paniculata and/or a Sidastrum species, in particular Sidastrum micranthum (syn: Sida micrantha) [lacuna] These compositions can furthermore comprise, as further additives, mild surfactants, oil bodies, emulsifiers, superfatting agents, pearlescent waxes, consistency modifiers, thickeners, polymers, silicone compounds, fats, waxes, lecithins, phospholipids, stabilisers, biogenic active ingredients, deodorants, antiperspirants, antidandruff agents, film formers, swelling agents, UV light-protection factors, antioxidants, hydrotropic agents, preservatives, insect repellents, self-tanning agents, tyrosine inhibitors, solubilisers, perfume oils, dyes and the like.

In a variant of the invention, the compositions are preferably compositions for topical application, for example cosmetic or dermatological formulations. In this case, the compositions comprise a cosmetically or dermatologically suitable carrier and, depending on the desired property profile, optionally further suitable ingredients.

Preferred compositions having light-protection properties comprise at least one dibenzoylmethane derivative. Of the dibenzoylmethane derivatives to which the present invention specifically relates, mention may made, in particular, of:
2-methyldibenzoylmethane,
4-methyldibenzoylmethane,
4-isopropyldibenzoylmethane,
4-tert-butyldibenzoylmethane,
2,4-dimethyldibenzoylmethane,
2,5-dimethyldibenzoylmethane,
4,4′-diisopropyldibenzoylmethane,
4,4′-methoxy-tert-butyldibenzoylmethane,
2-methyl-5-isopropyl-4′-methoxydibenzoylmethane,
2-methyl-5-tert-butyl-4′-methoxydibenzoylmethane,
2,4-dimethyl-4′-methoxydibenzoylmethane and
2,6-dimethyl-4-tert-butyl-4′-methoxydibenzoylmethane,
   this list being non-restrictive.

Of the above-mentioned dibenzoylmethane derivatives, particular preference is given in accordance with the invention to 4,4′-methoxy-tert-butyldibenzoylmethane and especially 4,4′-methoxy-tert-butyldibenzoylmethane, which is commercially available under the trade name Eusolex® 9020 from Merck, this filter conforming to the following structural formula:

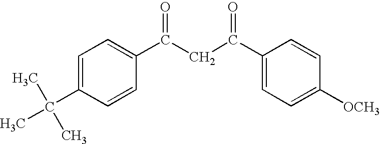

A further dibenzoylmethane derivative which is preferred in accordance with the invention is 4-isopropyldibenzoylmethane.

Further preferred compositions having light-protection properties comprise at least one benzophenone or benzophenone derivative, such as, particularly preferably, 2-hydroxy-4-methoxybenzophenone (for example Eusolex® 4360) or 2-hydroxy-4-methoxybenzophenone-5-sulfonic acid and the sodium salt thereof (for example Uvinul® MS-40).

The dibenzoylmethane derivative(s) or the benzophenone derivative(s) may be present in the compositions according to the invention in proportions which are generally in the range from 0.1 to 10% by weight and preferably in proportions which are in the range from 0.3 to 5% by weight, where these proportions are based on the total weight of the composition.

It may furthermore be preferred in accordance with the invention for the compositions to comprise further inorganic UV filters. Preference is given here both to those from the group consisting of titanium dioxides, such as, for example, coated titanium dioxide (for example Eusolex® T-2000, Eusolex® T-AQUA), zinc oxides (for example Sachtotec®), iron oxides, also cerium oxides. These inorganic UV filters are generally incorporated into cosmetic compositions in an amount of 0.5 to 20 percent by weight, preferably 2-10%. In particular, it may be preferred here for a nanoparticulate UV protectant according to the invention to be present in one phase in emulsions and a further inorganic UV filter to be present in the other phase.

In a further, likewise preferred embodiment of the present invention, the composition according to the invention comprises at least one self-tanning agent.

Advantageous self-tanning agents which can be employed are, inter glia:

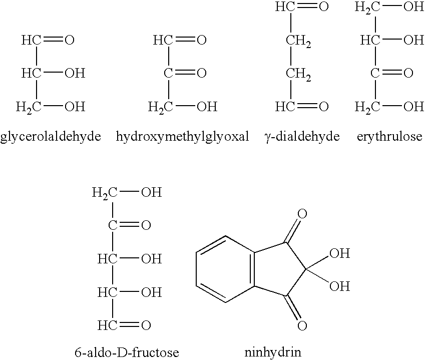

Mention should also be made of 5-hydroxy-1,4-naphthoquinone (juglone), which is extracted from the shells of fresh walnuts

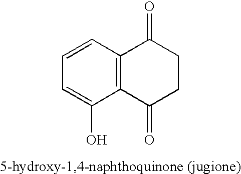

and 2-hydroxy-1,4-naphthoquinone (lawsone), which occurs in henna leaves.

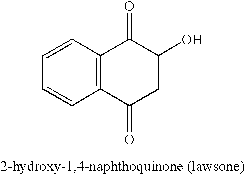

Very particular preference is given to 1,3-dihydroxyacetone (DHA), a trifunctional sugar which occurs in the human body, and derivatives thereof.

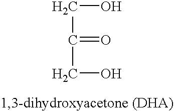

Furthermore, the compositions according to the invention may also comprise dyes and coloured pigments. The dyes and coloured pigments can be selected from the corresponding positive list in the German Cosmetics Regulation or the EC list of cosmetic colorants. In most cases, they are identical with the dyes approved for foods. Advantageous coloured pigments are, for example, titanium dioxide, mica, iron oxides (for example Fe₂O₃, Fe₃O₄, FeO(OH)) and/or tin oxide. Advantageous dyes are, for example, carmine, Berlin Blue, Chromium Oxide Green, Ultramarine Blue and/or Manganese Violet. It is particularly advantageous to select the dyes and/or coloured pigments from the following list. The Colour Index numbers (CINs) are taken from the Rowe Colour Index, 3rd Edition, Society of Dyers and Colourists, Bradford, England, 1971.

| Chemical or other name | CIN | Colour |
|---|---|---|
| Pigment Green | 10006 | green |
| Acid Green 1 | 10020 | Green |
| 2,4-Dinitrohydroxynaphthalene-7-sulfonic acid | 10316 | Yellow |
| Pigment Yellow 1 | 11680 | Yellow |
| Pigment Yellow 3 | 11710 | Yellow |
| Pigment Orange 1 | 11725 | Orange |
| 2,4-Dihydroxyazobenzene | 11920 | Orange |
| Solvent Red 3 | 12010 | Red |
| 1-(2′-Chloro-4′-nitro-1′-phenylazo)-2-hydroxynaphthalene | 12085 | Red |
| Pigment Red 3 | 12120 | Red |
| Ceres Red; Sudan Red; Fat Red G | 12150 | Red |
| Pigment Red 112 | 12370 | Red |
| Pigment Red 7 | 12420 | Red |
| Pigment Brown 1 | 12480 | Brown |
| 4-(2′-Methoxy-5′sulfonyldiethylamide-1′-phenylazo)-3-hydroxy- | 12490 | Red |
| 5″-chloro-2″,4″-dimethoxy2-naphthanilide | | |
| Disperse Yellow 16 | 12700 | Yellow |
| 1-(4-Sulfo-1-phenylazo)-4-aminobenzene-5-sulfonic acid | 13015 | Yellow |
| 2,4-Dihydroxyazobenzene-4′-sulfonic acid | 14270 | Orange |
| 2-(2,4-Dimethylphenylazo-5-sulfonyl)-1-hydroxynaphthalene- | 14700 | Red |
| 4-sulfonic acid | | |
| 2-(4-Sulfo-1-naphthylazo)-1-naphthol-4-sulfonic acid | 14720 | Red |
| 2-(6-Sulfo-2,4-xylylazo)-1-naphthol-5-sulfonic acid | 14815 | Red |
| 1-(4′-Sulfophenylazo)-2-hydroxynaphthalene | 15510 | Orange |
| 1-(2-Sulfonyl-4-chloro-5-carboxy-1-phenylazo)-2-hydroxy- | 15525 | Red |
| naphthalene | | |
| 1-(3-Methylphenylazo-4-sulfonyl)-2-hydroxynaphthalene | 15580 | Red |
| 1-(4′,(8′)-Sulfonylnaphthylazo)-2-hydroxynaphthalene | 15620 | Red |
| 2-Hydroxy-1,2′-azonaphthalene-1′-sulfonic acid | 15630 | Red |
| 3-Hydroxy-4-phenylazo-2-naphthylcarboxylic acid | 15800 | Red |
| 1-(2-Sulfo-4-methyl-1-phenylazo)-2-naphthylcarboxylic acid | 15850 | Red |
| 1-(2-Sulfo-4-methyl-5-chloro-1-phenylazo)-2-hydroxy- | 15865 | Red |
| naphthalene-3-carboxylic acid | | |
| 1-(2-Sulfo-1-naphthylazo)-2-hydroxynaphthalene-3-carboxylic | 15880 | red |
| acid | | |
| 1-(3-Sulfo-1-phenylazo)-2-naphthol-6-sulfonic acid | 15980 | Orange |
| 1-(4-Sulfo-1-phenylazo)-2-naphthol-6-sulfonic acid | 15985 | Yellow |
| Allura Red | 16035 | Red |
| 1-(4-Sulfo-1-naphthylazo)-2-naphthol-3,6-disulfonic acid | 16185 | Red |
| Acid Orange 10 | 16230 | Orange |
| 1-(4-Sulfo-1-naphthylazo)-2-naphthol-6,8-disulfonic acid | 16255 | Red |
| 1-(4-Sulfo-1-naphthylazo)-2-naphthol-3,6,8-trisulfonic acid | 16290 | Red |
| 8-Amino-2-phenylazo-1-naphthol-3,6-disulfonic acid | 17200 | Red |
| Acid Red 1 | 18050 | Red |
| Acid Red 155 | 18130 | Red |
| Acid Yellow 121 | 18690 | Yellow |
| Acid Red 180 | 18736 | Red |
| Acid Yellow 11 | 18820 | Yellow |
| Acid Yellow 17 | 18965 | Yellow |
| 4-(4-Sulfo-1-phenylazo)-1-(4-sulfophenyl)-5-hydroxy- | 19140 | Yellow |
| pyrazolone-3-carboxylic acid | | |
| Pigment Yellow 16 | 20040 | Yellow |
| 2,6-(4′-Sulfo-2″,4″-dimethyl)bisphenylazo)1,3-dihydroxy- | 20170 | Orange |
| benzene | | |
| Acid Black 1 | 20470 | Black |
| Pigment Yellow 13 | 21100 | Yellow |
| Pigment Yellow 83 | 21108 | Yellow |
| Solvent Yellow | 21230 | Yellow |
| Acid Red 163 | 24790 | Red |
| Acid Red 73 | 27290 | Red |
| 2-[4′-(4″-Sulfo-1″-phenylazo)-7′-sulfo-1′-naphthylazo]-1- | 27755 | black |
| hydroxy-7-aminonaphthalene-3,6-disulfonic acid | | |
| 4-[4″-Sulfo-1″-phenylazo)-7′-sulfo-1′-naphthylazo]-1-hydroxy- | 28440 | Black |
| 8-acetylaminonaphthalene-3,5-disulfonic acid | | |
| Direct Orange 34, 39, 44, 46, 60 | 40215 | Orange |
| Food Yellow | 40800 | Orange |
| trans-β-Apo-8′-carotene aldehyde (C₃₀) | 40820 | Orange |
| trans-Apo-8′-carotinic acid (C₃₀) ethyl ester | 40850 | Orange |
| Canthaxanthine | 40850 | Orange |
| Acid Blue 1 | 42045 | Blue |
| 2,4-Disulfo-5-hydroxy-4′-4″-bis(diethylamino)triphenylcarbinol | 42051 | Blue |
| 4-[(-4-N-Ethyl-p-sulfobenzylamino)phenyl-(4-hydroxy-2-sulfo- | 42053 | Green |
| phenyl)(methylene)-1-(N-ethylN-p-sulfobenzyl)-2,5-cyclo- | | |
| hexadienimine] | | |
| Acid Blue 7 | 42080 | Blue |
| (N-Ethyl-p-sulfobenzylamino)phenyl-(2-sulfophenyl)methylene- | 42090 | Blue |
| (N-ethyl-N-p-sulfobenzyl)Δ^{2,5-}cyclohexadienimine | | |
| Acid Green 9 | 42100 | Green |
| Diethyldisulfobenzyldi-4-amino-2-chlorodi-2-methylfuchsonimmonium | 42170 | Green |
| Basic Violet 14 | 42510 | Violet |
| Basic Violet 2 | 42520 | Violet |
| 2′-Methyl-4′-(N-ethyl-N-m-sulfobenzyl)amino-4″-(N-diethyl)- | 42735 | Blue |
| amino-2-methyl-N-ethylN-m-sulfobenzylfuchsonimmonium | | |
| 4′-(N-Dimethyl)amino-4″-(N-phenyl)aminonaphtho-N- | 44045 | Blue |
| dimethylfuchsonimmonium | | |
| 2-Hydroxy-3,6-disulfo-4,4′-bisdimethylaminonaphthofuchsonimmonium | 44090 | Green |
| Acid Red 52 | 45100 | Red |
| 3-(2′-Methylphenylamino)-6-(2′-methyl-4′-sulfophenylamino)-9- | 45190 | Violet |
| (2″-carboxyphenyl)xanthenium salt | | |
| Acid Red 50 | 45220 | Red |
| Phenyl-2-oxyfluorone-2-carboxylic acid | 45350 | yellow |
| 4,5-Dibromofluorescein | 45370 | Orange |
| 2,4,5,7-Tetrabromofluorescein | 45380 | Red |
| Solvent Dye | 45396 | Orange |
| Acid Red 98 | 45405 | Red |
| 3′,4′,5′,6′-Tetrachloro-2,4,5,7-tetrabromofluorescein | 45410 | Red |
| 4,5-Diiodofluorescein | 45425 | Red |
| 2,4,5,7-Tetraiodofluorescein | 45430 | Red |
| Quinophthalone | 47000 | Yellow |
| Quinophthalonedisulfonic acid | 47005 | Yellow |
| Acid Violet 50 | 50325 | Violet |
| Acid Black 2 | 50420 | Black |
| Pigment Violet 23 | 51319 | Violet |
| 1,2-Dioxyanthraquinone, calcium/aluminium complex | 58000 | Red |
| 3-Oxypyrene-5,8,10-sulfonic acid | 59040 | Green |
| 1-Hydroxy-4-N-phenylaminoanthraquinone | 60724 | Violet |
| 1-Hydroxy-4-(4′-methylphenylamino)anthraquinone | 60725 | Violet |
| Acid Violet 23 | 60730 | Violet |
| 1,4-Di(4′-methylphenylamino)anthraquinone | 61565 | Green |
| 1,4-Bis(o-sulfo-p-toluidino)anthraquinone | 61570 | Green |
| Acid Blue 80 | 61585 | Blue |
| Acid Blue 62 | 62045 | Blue |
| N,N′-Dihydro-1,2,1′,2′-anthraquinonazine | 69800 | Blue |
| Vat Blue 6; Pigment Blue 64 | 69825 | Blue |
| Vat Orange 7 | 71105 | orange |
| Indigo | 73000 | Blue |
| Indigodisulfonic acid | 73015 | Blue |
| 4,4′-Dimethyl-6,6′-dichlorothioindigo | 73360 | Red |
| 5,5′Dichloro-7,7′-dimethylthioindigo | 73385 | violet |
| Quinacridone Violet 19 | 73900 | violet |
| Pigment Red 122 | 73915 | Red |
| Pigment Blue 16 | 74100 | blue |
| Phthalocyanines | 74160 | blue |
| Direct Blue 86 | 74180 | blue |
| Chlorinated phthalocyanines | 74260 | green |
| Natural Yellow 6, 19; Natural Red 1 | 75100 | yellow |
| Bixin, Nor-Bixin | 75120 | orange |
| Lycopene | 75125 | yellow |
| trans-alpha-, -beta- or -gamma-Carotene | 75130 | orange |
| Keto and/or hydroxyl derivatives of carotene | 75135 | yellow |
| Guanine or pearlescent agent | 75170 | white |
| 1,7-Bis(4-hydroxy-3-methoxyphenyl)1,6-heptadiene-3,5-dione | 75300 | yellow |
| Complex salt (Na, Al, Ca) of carminic acid | 75470 | Red |
| Chlorophyll a and b; copper compounds of chlorophylls and | 75810 | green |
| chlorophyllines | | |
| Aluminium | 77000 | white |
| Aluminium hydroxide | 77002 | white |
| Water-containing aluminium silicates | 77004 | white |
| Ultramarine | 77007 | blue |
| Pigment Red 101 and 102 | 77015 | Red |
| Barium sulfate | 77120 | white |
| Bismuth oxychloride and mixtures thereof with mica | 77163 | white |
| Calcium carbonate | 77220 | white |
| Calcium sulfate | 77231 | white |
| Carbon | 77266 | black |
| Pigment Black 9 | 77267 | black |
| Carbo medicinalis vegetabilis | 77268:1 | black |
| Chromium oxide | 77288 | green |
| Chromium oxide, water-containing | 77278 | green |
| Pigment Blue 28, Pigment Green 14 | 77346 | green |
| Pigment Metal 2 | 77400 | brown |
| Gold | 77480 | brown |
| Iron oxides and hydroxides | 77489 | orange |
| Iron oxide | 77491 | red |
| Iron oxide hydrate | 77492 | yellow |
| Iron oxide | 77499 | black |
| Mixtures of iron(II) and iron(III) hexacyanoferrate | 77510 | blue |
| Pigment White 18 | 77713 | white |
| Manganese ammonium diphosphate | 77742 | violet |
| Manganese phosphate; Mn₃(PO₄)₂•7H₂O | 77745 | red |
| Silver | 77820 | white |
| Titanium dioxide and mixtures thereof with mica | 77891 | white |
| Zinc oxide | 77947 | white |
| 6,7-Dimethyl-9-(1′-D-ribityl)isoalloxazine, lactoflavin | | yellow |
| Sugar dye | | brown |
| Capsanthin, capsorubin | | orange |
| Betanin | | red |
| Benzopyrylium salts, anthocyans | | red |
| Aluminium, zinc, magnesium and calcium stearate | | white |
| Bromothymol Blue | | blue |

It may furthermore be favourable to select, as dye, one or more substances from the following group:

2,4-dihydroxyazobenzene, 1-(2′-chloro-4′-nitro-1′phenylazo)-2-hydroxynaphthalene, Ceres Red, 2-(4-sulfo-1-naphthylazo)-1-naphthol-4-sulfonic acid, the calcium salt of 2-hydroxy-1,2′-azonaphthalene-1′-sulfonic acid, the calcium and barium salts of 1-(2-sulfa-4-methyl-1-phenylazo)-2-naphthylcarboxylic acid, the calcium salt of 1-(2-sulfo-1-naphthylazo)-2-hydroxynaphthalene-3-carboxylic acid, the aluminium salt of 1-(4-sulfo-1-phenylazo)-2-naphthol-6-sulfonic acid, the aluminium salt of 1-(4-sulfo-1-naphthylazo)-2-naphthol-3,6-disulfonic acid, 1-(4-sulfo-1-naphthylazo)-2-naphthol-6,8-disulfonic acid, the aluminium salt of 4-(4-sulfo-1-phenylazo)-2-(4-sulfophenyl)-5-hydroxypyrazolone-3-carboxylic acid, the aluminium and zirconium salts of 4,5-dibromofluorescein, the aluminium and zirconium salts of 2,4,5,7-tetrabromofluorescein, 3′,4′,5′,6′-tetrachloro-2,4,5,7-tetrabromofluorescein and its aluminium salt, the aluminium salt of 2,4,5,7-tetraiodofluorescein, the aluminium salt of quinophthalonedisulfonic acid, the aluminium salt of indigodisulfonic acid, red and black iron oxide (CIN: 77 491 (red) and 77 499 (black)), iron oxide hydrate (CIN: 77492), manganese ammonium diphosphate and titanium dioxide.

Also advantageous are oil-soluble natural dyes, such as, for example, paprika extract, β-carotene or cochineal.

Also advantageous for the purposes of the present invention are gel creams comprising pearlescent pigments. Particular preference is given to the types of pearlescent pigment listed below:
1. Natural pearlescent pigments, such as, for example,
   a) “pearl essence” (guanine/hypoxanthine mixed crystals from fish scales) and
   b) “mother-of-pearl” (ground mussel shells)
2. Monocrystalline pearlescent pigments, such as, for example, bismuth oxychloride (BiOCl)
3. Layered substrate pigments: for example mica/metal oxide

The basis for pearlescent pigments is formed by, for example, pulverulent pigments or castor oil dispersions of bismuth oxychloride and/or titanium dioxide as well as bismuth oxychloride and/or titanium dioxide on mica. The lustre pigment listed under CIN 77163, for example, is particularly advantageous.

Also advantageous are, for example, the following pearlescent pigment types based on mica/metal oxide:

| Group | Coating/layer thickness | Colour |
|---|---|---|
| Silver-white pearlescent | TiO₂: 40-60 nm | silver |
| pigments | | |
| Interference pigments | TiO₂: 60-80 nm | yellow |
| | TiO₂: 80-100 nm | red |
| | TiO₂: 100-140 nm | blue |
| | TiO₂: 120-160 nm | green |
| Coloured lustre pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold shades |
| | TiO₂/Cr₂O₃ | green |
| | TiO₂/Berlin Blue | dark blue |

Particular preference is given to, for example, the pearlescent pigments available from Merck under the trade names Timiron, Colorona or Dichrona.

The list of the said pearlescent pigments is of course not intended to be limiting. Pearlescent pigments which are advantageous for the purposes of the present invention can be obtained by numerous routes known per se. For example, other substrates apart from mica can also be coated with further metal oxides, such as, for example, silica and the like. For example, TiO₂- and Fe₂O₃-coated SiO₂ particles (“Ronasphere” grades), which are marketed by Merck and are particularly suitable for the optical reduction of fine wrinkles, are advantageous.

it may additionally be advantageous to completely omit a substrate such as mica. Particular preference is given to pearlescent pigments prepared using SiO₂. Such pigments, which may additionally also have goniochromatic effects, are available, for example, from BASF under the trade name Sicopearl Fantastico.

It may also be advantageous to employ Engelhard/Mearl pigments based on calcium sodium borosilicate coated with titanium dioxide. These are available under the name Reflecks. Due to their particle size of 40-80 μm, they have a glitter effect in addition to the colour.

Also particularly advantageous are effect pigments available from Flora Tech under the trade name Metasomes Standard/Glitter in various colours (yellow, red, green, blue). The glitter particles here are in the form of mixtures with various assistants and dyes (such as, for example, the dyes with the Colour Index (CI) numbers 19140, 77007, 77289, 77491).

The dyes and pigments can be in individual form or in the form of a mixture and mutually coated with one another, with different colour effects generally being caused by different coating thicknesses. The total amount of dyes and colouring pigments is advantageously selected from the range from, for example, 0.1% by weight to 30% by weight, preferably 0.5 to 15% by weight, in particular 1.0 to 10% by weight, in each case based on the total weight of the compositions.

The compositions according to the invention may of course comprise one or more hydrophilic or lipophilic sunscreen filters which are effective in the UV-A region and/or UV-B region and/or IR and/or VIS region (absorbers). These filters can be selected, in particular, from cinnamic acid derivatives, salicylic acid derivatives, camphor derivatives, triazine derivatives, β,β-diphenyl acrylate derivatives, p-aminobenzoic acid derivatives and polymeric filters and silicone filters, which are described in the application WO 93/04665. Further examples of organic filters are indicated in patent application EP-A 0 487 404.

In principle, all UV filters are suitable. Particular preference is given to UV filters whose physiological acceptability has already been demonstrated. Both for UVA and UVB filters, there are many proven substances known from the specialist literature, for example

benzylidenecamphor derivatives, such as 3-(4′-methylbenzylidene)-di-camphor (for example Eusolex® 6300), 3-benzylidenecamphor (for example Mexoryl® SD), polymers of N-{(2 and 4)-[(2-oxoborn-3-ylidene)methyl]benzyl}acrylamide (for example Mexoryl® SW), N,N,N-trimethyl-4-(2-oxoborn-3-ylidenemethyl)anilinium methylsulfate (for example Mexoryl® SK) or (2-oxoborn-3-ylidene)toluene-4-sulfonic acid (for example Mexoryl® SL),
methoxycinnamic acid esters, such as octyl methoxycinnamate (for example Eusolex® 2292), isopentyl 4-methoxycinnamate, for example as a mixture of the isomers (for example Neo Heliopan® E 1000),
salicylate derivatives, such as 2-ethylhexyl salicylate (for example Eusolex® OS), 4-isopropylbenzyl salicylate (for example Megasol®) or 3,3,5-trimethylcyclohexyl salicylate (for example Eusolex® HMS),
4-aminobenzoic acid and derivatives, such as 4-aminobenzoic acid, 2-ethylhexyl 4-(dimethylamino)benzoate (for example Eusolex® 6007), ethoxylated ethyl 4-aminobenzoate (for example Uvinul® P25),
phenylbenzimidazolesulfonic acids, such as 2-phenylbenzimidazole-5-sulfonic acid and potassium, sodium and triethanolamine salts thereof (for example Eusolex® 232), 2,2-(1,4-phenylene)bisbenzimidazole-4,6-disulfonic acid and salts thereof (for example Neoheliopan® AP) or 2,2-(1,4-phenylene)bisbenzimidazole-6-sulfonic acid;
and further substances, such as
2-ethylhexyl 2-cyano-3,3-diphenylacrylate (for example Eusolex® OCR),
3.3′-(1,4-phenylenedimethylene)bis(7,7-dimethyl-2-oxobicyclo[2.2.1]hept-1-ylmethanesulfonic acid and salts thereof (for example Mexoryl® SX) and
2,4,6-trianilino-(p-carbo-2′-ethylhexyl-1′-oxy)-1,3,5-triazine (for example Uvinul® T 150)
hexyl 2-(4-diethylamino-2-hydroxybenzoyl)benzoate (for example Uvinul®UVA Plus, BASF).

The compounds mentioned in the list should only be regarded as examples. It is of course also possible to use other UV filters. In particular, organic particulate UV filters, as described, for example, in patent application WO 99/66896, can advantageously be employed.

These organic UV filters are generally incorporated into cosmetic formulations in an amount of 0.5 to 20 percent by weight, preferably 1-10%.

Further suitable organic UV filters are, for example,
2-(2H-benzotriazol-2-yl)-4-methyl-6-(2-methyl-3-(1,3,3,3-tetramethyl-1-(trimethylsilyloxy)disiloxanyl)propyl)phenol (for example Silatrizole®),
2-ethylhexyl 4.4′-[(6-[4-((1,1-dimethylethyl)aminocarbonyl)phenylamino]-1,3,5-triazine-2,4-diyl)diimino]bis(benzoate) (for example Uvasorb® HEB),
α-(trimethylsilyl)-ω-[trimethylsilyl)oxy]poly[oxy(dimethyl [and approximately 6% of methyl[2-[p-[2,2-bis(ethoxycarbonyl]vinyl]phenoxy]-1-methyleneethyl] and approximately 1.5% of methyl[3-[p-[2,2-bis(ethoxycarbonyl)vinyl])phenoxy)-propenyl) and 0.1 to 0.4% of (methylhydrogen]silylene]] (n˜60) (CAS No. 207 574-74-1)
2.2′-methylenebis(6-(2H-benzotriazol-2-yl)-4-(1,1,3,3-tetramethylbutyl)phenol) (CAS No. 103 597-45-1)
2.2′-(1,4-phenylene)bis(1H-benzimidazole-4,6-disulfonic acid, monosodium salt) (CAS No. 180 898-37-7) and
2,4-bis{[4-(2-ethylhexyloxy)-2-hydroxy]phenyl}-6-(4-methoxyphenyl)-1,3,5-triazine (CAS No. 103 597-45-, 187 393-00-6).
2-ethylhexyl 4.4′-[(6-[4-((1,1-dimethylethyl)aminocarbonyl)phenylamino]-1,3,5-triazine-2,4-diyl)diimino]bis(benzoate) (for example Uvasorb® HEB),

Organic UV filters are generally incorporated into cosmetic formulations in a total amount of 0.5 to 20 percent by weight, preferably 1-15%.

Preferred compounds having UV-filtering properties are 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and potassium, sodium and triethanolamine salts thereof.

Preferred compositions may also comprise compounds of the formula I

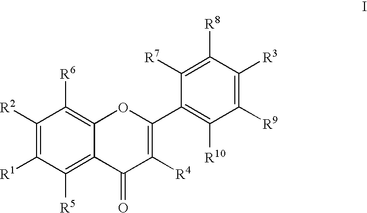

where R¹ and R² are selected from
   H
   and OR¹¹, where OR¹¹, independently of one another, stands for
      OH
      straight-chain or branched C₁- to C₂₀-alkoxy groups,
      straight-chain or branched C₃- to C₂₀-alkenyloxy groups,
      straight-chain or branched C₁- to C₂₀-hydroxyalkoxy groups,
      where the hydroxyl group(s) may be bonded to a primary or secondary carbon atoms of the chain and furthermore the alkyl chain may also be interrupted by oxygen, and/or
      C₃- to C₁₀-cycloalkoxy groups and/or C₃- to C₁₂-cycloalkenyloxy groups, where the rings may each also be bridged by —(CH₂)ₙ— groups, where n=1 to 3, and/or
      mono- and/or oligoglycosyl radicals,
with the proviso that at least one radical from R¹ and R² stands for OR₁₁, and R³ stands for a radical OR¹¹ and
R⁴ to R⁷ and R¹⁰ may be identical or different and, independently of one another, stand for
   H
   straight-chain or branched C₁- to C₂₀-alkyl groups,
   straight-chain or branched C₃- to C₂₀-alkenyl groups,
   straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
   C₃- to C₁₀-cycloalkyl groups and/or C₃ to C₁₂-cycloalkenyl groups, where the rings may each also be bridged by —(CH₂)ₙ— groups, where n=1 to 3, and
R⁸ and R⁹ may be identical or different and, independently of one another, stand for
   H
   OR¹¹
   straight-chain or branched C₁- to C₂₀-alkyl groups,
   straight-chain or branched C₃- to C₂₀-alkenyl groups,
   straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
   C₃- to C₁₀-cycloalkyl groups and/or C₃ to C₁₂-cycloalkenyl groups, where the rings may each also be bridged by —(CH₂)ₙ— groups, where n=1 to 3.

Of the flavonoids of the formula I to be employed in accordance with the invention, broad-band UV filters [lacuna] other likewise preferred compounds of the formula I exhibit an absorption maximum in the boundary region between UV-B and UV-A radiation. As UV-A-II filters, they therefore advantageously supplement the absorption spectrum of commercially available UV-B and UV-A-I filters. Preferred compositions according to the invention having light-protection properties comprise at least one compound of the formula I, where R³ stands for
OH or
straight-chain or branched to C₁- to C₂₀-alkoxy groups, preferably methoxy, ethoxy or ethylhexyloxy, or
mono- and/or oligoglycosyl radicals, preferably glucosyl radicals, and R¹ and/or R² preferably stand for
OH or
straight-chain or branched C₁- to C₂₀-alkoxy groups, preferably methoxy, ethoxy or ethylhexyloxy, or
mono- and/or oligoglycosyl radicals, preferably glucosyl radicals.

These preferred compounds are distinguished by particularly intense UV absorption. It has been found that the intensity of the UV absorption is particularly high if R³ stands for straight-chain or branched C₁- to C₂₀-alkoxy groups, preferably methoxy, ethoxy or ethylhexyloxy, and R⁸ and R⁹ are identical and stand for H or straight-chain or branched C₁- to C₂₀-alkoxy groups, preferably methoxy, ethoxy or ethylhexyloxy. Particular preference is therefore given in accordance with the invention to compositions having light-protection properties comprising at least one compound of the formula I which is characterised in that R³ stands for straight-chain or branched C₁- to C₂₀-alkoxy groups, preferably methoxy, ethoxy or ethylhexyloxy, and R⁸ and R⁹ are identical and stand for H or straight-chain or branched C₁- to C₂₀-alkoxy groups, preferably methoxy, ethoxy or ethylhexyloxy. It is particularly preferred here if R⁸ and R⁹ stand for H. The compounds of the formula I are typically employed in accordance with the invention in amounts of 0.01 to 20% by weight, preferably in amounts of 0.5% by weight to 10% by weight and particularly preferably in amounts of 1 to 8% by weight. The person skilled in the art is presented with absolutely no difficulties at all in correspondingly selecting the amounts depending on the intended light protection factor of the composition.

it may furthermore be preferred in accordance with the invention for the compositions to comprise inorganic UV filters. Preference is given here both to those from the group consisting of titanium dioxides, such as, for example, coated titanium dioxide (for example Eusolex® T-2000, Eusolex® T-AQUA, Eusolex® T-AVO, Eusolex® T-Oleo), zinc oxides (for example Sachtotec®), iron oxides, also cerium oxides. These inorganic UV filters are generally incorporated into cosmetic compositions in an amount of 0.5 to 20 percent by weight, preferably 2-10%. Combination of one or more nanoparticulate UV protectants with further UV filters enables the protective action against harmful effects of UV radiation to be optimised. Optimised compositions may comprise, for example, the combination of the organic filters 4′-methoxy-6-hydroxyflavone with 1-(4-tert-butylphenyl)-3-(4-methoxyphenyl)propane-1,3-dione and 3-(4′-methylbenzylidene)-dl-camphor with nanoparticulate titanium dioxide.

All the said UV filters including the compounds of the formula I can also be employed in encapsulated form. In particular, it is advantageous to employ organic UV filters in encapsulated form. In detail, the following advantages arise:
The hydrophilicity of the capsule wall can be set independently of the solubility of the UV filter. Thus, for example, it is also possible to incorporate hydrophobic UV filters into purely aqueous compositions. In addition, the oily impression on application of the composition comprising hydrophobic UV filters, which is frequently regarded as unpleasant, is suppressed.
Certain UV filters, in particular dibenzoylmethane derivatives, exhibit only reduced photostability in cosmetic compositions. Encapsulation of these filters or compounds which impair the photostability of these filters, such as, for example, cinnamic acid derivatives, enables the photostability of the entire composition to be increased.
Skin penetration by organic UV filters and the associated potential for irritation on direct application to the human skin is repeatedly being discussed in the literature. The encapsulation of the corresponding substances which is proposed here suppresses this effect.
In general, encapsulation of individual UV filters or other ingredients enables preparation problems caused by the interaction of individual composition constituents with one another, such as crystallisation processes, precipitation and agglomeration, to be avoided since the interaction is suppressed.

It may therefore be preferred in accordance with the invention for one or more of the compounds of the formula I or the above-mentioned UV filters to be in encapsulated form. It is advantageous here for the capsules to be so small that they cannot be observed with the naked eye. In order to achieve the above-mentioned effects, it is furthermore necessary for the capsules to be sufficiently stable and the encapsulated active ingredient (UV filter) only to be released to the environment to a small extent, or not at all. Suitable capsules can have walls of inorganic or organic polymers. For example, U.S. Pat. No. 6,242,099 B1 describes the production of suitable capsules with walls of chitin, chitin derivatives or polyhydroxylated polyamines. Capsules particularly preferably to be employed in accordance with the invention have walls which can be obtained by a sol-gel process, as described in the applications WO 00/09652, WO 00/72806 and WO 00/71084. Preference is in turn given here to capsules whose walls are built up from silica gel (silica; undefined silicon oxide hydroxide). The production of corresponding capsules is known to the person skilled in the art, for example from the cited patent applications, whose contents expressly also belong to the subject-matter of the present application. The capsules are preferably present in compositions according to the invention in amounts which ensure that the encapsulated UV filters are present in the composition in the above-indicated amounts.

If the compositions according to the invention comprise compounds of the formula containing free hydroxyl groups, they additionally, besides the properties described, exhibit an action as antioxidant and/or free-radical scavenger. Preference is therefore also given to compositions having light-protection properties comprising at least one compound of the formula I which is characterised in that at least one of the radicals R¹ to R³ stands for OH, preferably with at least one of the radicals R¹ or R² standing for OH.

In order that the compounds of the formula I or the extracts according to the invention obtained from. Waltheria paniculata or pure substances obtained from a Waltheria species are able to develop their positive action as free-radical scavengers particularly well on the skin, it may be preferred to allow the compounds/extracts to penetrate into deeper skin layers. Several possibilities are available for this purpose. Firstly, the compounds/extracts can have an adequate lipophilicity in order to be able to penetrate through the outer skin layer into epidermal layers. As a further possibility, corresponding transport agents, for example liposomes, which enable transport of the compounds/extracts through the outer skin layers may also be provided in the composition. Finally, systemic transport of the compounds of the formula I is also conceivable. The composition is then designed, for example, in such a way that it is suitable for oral administration.

In general, the substances of the formula I act as free-radical scavengers. Free radicals of this type are not generated only by sunlight, but instead are formed under various conditions. Examples are anoxia, which blocks the flow of electrons upstream of the cytochrome oxidases and causes the formation of superoxide free-radical anions; inflammation associated, inter alia, with the formation of superoxide anions by the membrane NADPH oxidase of the leucocytes, but also associated with the formation (through disproportionation in the presence of iron(II) ions) of the hydroxyl free radicals and other reactive species which are normally involved in the phenomenon of phagocytosis; and lipid autoxidation, which is generally initiated by a hydroxyl free radical and produces lipidic alkoxy free radicals and hydroperoxides.

It is assumed that preferred compounds/extracts according to the invention also act as enzyme inhibitors. They are thought to inhibit histidine decarboxylase, protein kinases, elastase, aldose reductase and hyaluronidase, and therefore enable the intactness of the basic substance of vascular sheaths to be maintained. Furthermore, they are thought to inhibit catechol O-methyl transferase nonspecifically, causing the amount of available catecholamines and thus the vascular strength to be increased. Furthermore, they inhibit AMP phosphodiesterase, giving the substances potential for inhibiting thrombocyte aggregation. Owing to these properties, the compositions according to the invention are, in general, suitable for immune protection and for the protection of DNA and RNA. In particular, the compositions are suitable for the protection of DNA and RNA against oxidative attack, against free radicals and against damage due to radiation, in particular UV radiation. A further advantage of the compositions according to the invention is cell protection, in particular protection of Langerhans cells against damage due to the above-mentioned influences. The present invention also expressly relates to all these uses and to the use of the compounds/extracts according to the invention for the preparation of compositions which can be employed correspondingly.

In particular, preferred compositions according to the invention are also suitable for the treatment of skin diseases associated with a defect in keratinisation which affects differentiation and cell proliferation, in particular for the treatment of acne vulgaris, acne comedonica, polymorphic acne, acne rosaceae, nodular acne, acne conglobata, age-induced acne, acne which arises as a side effect, such as acne solaris, medicament-induced acne or acne professionalis, for the treatment of other defects in keratinisation, in particular ichthyosis, ichthyosiform states, Darier's disease, keratosis palmoplantaris, leucoplasia, leucoplasiform states, herpes of the skin and mucous membrane (buccal) (lichen), for the treatment of other skin diseases associated with a defect in keratinisation and which have an inflammatory and/or immunoallergic component and in particular all forms of psoriasis which affect the skin, mucous membranes and fingers and toenails, and psoriatic rheumatism and skin atopy, such as eczema or respiratory atopy, or hypertrophy of the gums, it furthermore being possible for the compounds to be used for some inflammation which is not associated with a defect in keratinisation, for the treatment of all benign or malignant excrescence of the dermis or epidermis, which may be of viral origin, such as verruca vulgaris, verruca plana, epidermodysplasia verruciform is, oral papillomatosis, papillomatosis florida, and excrescence which may be caused by UV radiation, in particular epithelioma baso-cellulare and epithelioma spinocellulare, for the treatment of other skin diseases, such as dermatitis bullosa and diseases affecting the collagen, for the treatment of certain eye diseases, in particular corneal diseases, for overcoming or combating light-induced skin ageing associated with ageing, for reducing pigmentation and keratosis actinica and for the treatment of all diseases associated with normal ageing or light-induced ageing, for the prevention or healing of wounds/scars of atrophy of the epidermis and/or dermis caused by locally or systemically applied corticosteroids and all other types of skin atrophy, for the prevention or treatment of defects in wound healing, for the prevention or elimination of stretch marks caused by pregnancy or for the promotion of wound healing, for combating defects in sebum production, such as hyperseborrhoea in acne or simple seborrhoea, for combating or preventing cancer-like states or pre-carcinogenic states, in particular promyelocytic leukaemia, for the treatment of inflammatory diseases, such as arthritis, for the treatment of all virus-induced diseases of the skin or other areas of the body, for the prevention or treatment of alopecia, for the treatment of skin diseases or diseases of other areas of the body with an immunological component, for the treatment of cardiovascular diseases, such as arteriosclerosis or hypertension, and of non-insulin-dependent diabetes, and for the treatment of skin problems caused by UV radiation.

The protective action against oxidative stress or against the effect of free radicals can be further improved if the compositions comprise one or more antioxidants.

In a preferred embodiment of the present invention, the composition is therefore a composition for the protection of body cells against oxidative stress, in particular for reducing skin ageing, characterised in that it preferably comprises one or more antioxidants.

There are many proven substances known from the specialist literature which can be used as antioxidants, for example amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotenoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and also (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate), and coniferyl benzoate of benzoin resin, rutinic acid and derivatives thereof, α-glycosyl rutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Mixtures of antioxidants are likewise suitable for use in the cosmetic compositions according to the invention. Known and commercial mixtures are, for example, mixtures comprising, as active ingredients, lecithin, L-(+)-ascorbyl palmitate and citric acid (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004).

The compositions according to the invention may comprise vitamins as further ingredients. The cosmetic compositions according to the invention preferably comprise vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active ingredient), thiamine (vitamin B₁), nicotinic acid (niacin), pyridoxine, pyridoxal, pyridoxamine (vitamin B₆), pantothenic acid, biotin, folic acid and cobalamine (vitamin B₁₂), particularly preferably vitamin A palmitate, vitamin C, DL-α-tocopherol, tocopherol E acetate, nicotinic acid, pantothenic acid and biotin.

The compositions according to the invention may in addition comprise further conventional skin-protecting or skin-care active ingredients. These can in principle be any active ingredients known to the person skilled in the art.

Particularly preferred active ingredients are pyrimidinecarboxylic acids and/or aryl oximes.

Pyrimidinecarboxylic acids occur in halophilic microorganisms and play a role in osmoregulation of these organisms (E. A. Galinski at al., Eur. J. Biochem., 149 (1985) pages 135-139). Of the pyrimidinecarboxylic acids, particular mention should be made here of ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid) and derivatives thereof. These compounds stabilise enzymes and other biomolecules in aqueous solutions and organic solvents. Furthermore, they stabilise, in particular, enzymes against denaturing conditions, such as salts, extreme pH values, surfactants, urea, guanidinium chloride and other compounds.

Ectoine and ectoine derivatives, such as hydroxyectoine, can advantageously be used in medicaments. In particular, hydroxyectoine can be employed for the preparation of a medicament for the treatment of skin diseases. Other areas of application of hydroxyectoine and other ectoine derivatives are typically in areas in which, for example, trehalose is used as additive. Thus, ectoine derivatives, such as hydroxyectoine, can be used as protectant in dried yeast and bacteria cells. Pharmaceutical products, such as non-glycosylated, pharmaceutically active peptides and proteins, for example t-PA, can also be protected with ectoine or its derivatives.

Of the cosmetic applications, particular mention should be made of the use of ectoine and ectoine derivatives for the care of aged, dry or irritated skin. Thus, European patent application EP-A-0 671 161 describes, in particular, that ectoine and hydroxyectoine are employed in cosmetic compositions, such as powders, soaps, surfactant-containing cleansing products, lipsticks, rouge, make-up, care creams and sunscreen preparations.

Preference is given here to the use of a pyrimidinecarboxylic acid of the following formula II

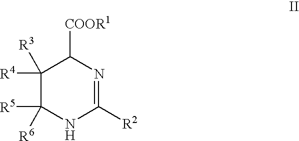

in which R¹ is a radical H or C1-8-alkyl, R² is a radical H or C1-4-alkyl, and R³, R⁴, R⁵ and R⁶ are each, independently of one another, a radical from the group consisting of H, OH, NH₂ and C1-4-alkyl. Preference is given to the use of pyrimidinecarboxylic acids in which R² is a methyl or ethyl group, and R¹ or R⁵ and R⁶ are H. Particular preference is given to the use of the pyrimidinecarboxylic acids ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid). In this case, the compositions according to the invention preferably comprise pyrimidinecarboxylic acids of this type in amounts of up to 15% by weight.

Of the aryl oximes, preference is given to the use of 2-hydroxy-5-methyllaurophenone oxime, which is also known as HMLO, LPO or F5. Its suitability for use in cosmetic compositions is disclosed, for example, in DE-A-41 16 123. Compositions which comprise 2-hydroxy-5-methyllaurophenone oxime are accordingly suitable for the treatment of skin diseases which are accompanied by inflammation. It is known that compositions of this type can be used, for example, for the therapy of psoriasis, various forms of eczema, irritative and toxic dermatitis, UV dermatitis and further allergic and/or inflammatory diseases of the skin and skin appendages. Compositions according to the invention which comprise aryl oximes, preferably 2-hydroxy-5-methyllaurophenone oxime, exhibit surprising antiinflammatory suitability. The compositions here preferably comprise 0.01 to 10% by weight of the aryl oxime, it being particularly preferred for the composition to comprise 0.05 to 5% by weight of aryl oxime.

All compounds or components described here that can be used in the compositions are either known and commercially available or can be synthesised by known processes.

Besides the compounds described here, the compositions according to the invention may also comprise at least one photostabiliser, preferably conforming to the formula III

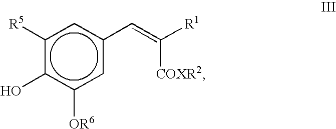

where
R¹ is selected from —C(O)CH₃, —CO₂R³, —C(O)NH₂ and —C(O)N(R⁴)₂;
X is O or NH;
R² stands for a linear or branched C₁₋₃₀-alkyl radical;
R³ stands for a linear or branched C₁₋₂₀-alkyl radical;
all R⁴, independently of one another, stand for H or linear or branched C₁₋₈-alkyl radicals;
R⁵ stands for H, a linear or branched C₁₋₈-alkyl radical or a linear or branched —O—C₁₋₈-alkyl radical; and
R⁶ stands for a C₁₋₈-alkyl radical,
   where the photostabiliser is particularly preferably bis(2-ethylhexyl) 2-(4-hydroxy-3,5-dimethoxybenzylidene)malonate. Corresponding photostabilisers and their preparation and use are described in International patent application WO 03/007906, the disclosure content of which expressly also belongs to the subject-matter of the present application.

The compositions according to the invention can be prepared by processes which are well known to the person skilled in the art, in particular by the processes which serve for the preparation of oil-in-water emulsions or water-in-oil emulsions.

The present invention furthermore relates to a process for the preparation of a composition which is characterised in that a compound/extract according to the invention is mixed with a cosmetically or dermatologically suitable carrier.

These compositions can be, in particular, in the form of simple or complex emulsions (O/W, W/O, O/W/O or W/O/W), such as creams, milks, gels or gel creams, powders and solid sticks, and they may, if desired, be formulated as aerosols and be in the form of foams or sprays. These compositions are preferably in the form of an O/W emulsion.

The cosmetic compositions according to the invention can be used as compositions for protection of the human epidermis or of the hair against UV radiation, as sunscreen compositions or make-up products.

It should be pointed out that in the formulations according to the invention which have a carrier of the oil-in-water emulsion type, the aqueous phase (which comprises, in particular, the hydrophilic filters) generally makes up 50 to 95% by weight and preferably 70 to 90% by weight, based on the formulation as a whole, the oil phase (which comprises, in particular, the lipophilic filters) makes up 5 to 50% by weight and preferably 10 to 30% by weight, based on the formulation as a whole, and the (co)emulsifier or (co)emulsifiers make(s) up 0.5 to 20% by weight and preferably 2 to 10% by weight, based on the formulation as a whole.

Suitable compositions are those for external use, for example in the form of a cream, lotion or gel or as a solution which can be sprayed onto the skin. Suitable for internal use are administration forms such as capsules, coated tablets, powders, tablet solutions or solutions.

Examples which may be mentioned of application forms of the compositions according to the invention are: solutions, suspensions, emulsions, PIT emulsions, pastes, ointments, gels, creams, lotions, powders, soaps, surfactant-containing cleansing preparations, oils, aerosols and sprays. Examples of other application forms are sticks, shampoos and shower products. Any desired customary carriers, auxiliaries and, if desired, further active ingredients may be added to the composition.

Preferred auxiliaries originate from the group consisting of preservatives, antioxidants, stabilisers, solubilisers, vitamins, colorants and odour improvers.

Ointments, pastes, creams and gels may comprise the customary carriers, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary carriers, for example lactose, talc, silica, aluminium hydroxide, calcium silicate and polyamide powder, or mixtures of these substances. Sprays may additionally comprise the customary propellants, for example chlorofluorocarbons, propane/butane or dimethyl ether.

Solutions and emulsions may comprise the customary carriers, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

Suspensions may comprise the customary carriers, such as liquid diluents, for example water, ethanol or propylene glycol, suspending agents, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary carriers, such as alkali metal salts of fatty acids, salts of fatty acid monoesters, fatty acid protein hydrolysates, isethionates, lanolin, fatty alcohol, vegetable oils, plant extracts, glycerol, sugars, or mixtures of these substances.

Surfactant-containing cleansing products may comprise the customary carriers, such as salts of fatty alcohol sulfates, fatty alcohol ether sulfates, sulfosuccinic acid monoesters, fatty acid protein hydrolysates, isethionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary carriers, such as synthetic oils, such as fatty acid esters, fatty alcohols, silicone oils, natural oils, such as vegetable oils and oily plant extracts, paraffin oils, lanolin oils, or mixtures of these substances.

Further typical cosmetic application forms are also lipsticks, lip-care sticks, mascara, eyeliner, eye shadow, rouge, powder make-up, emulsion make-up and wax make-up, and sunscreen, pre-sun and after-sun preparations.

The preferred composition forms according to the invention include, in particular, emulsions.

Emulsions according to the invention are advantageous and comprise, for example, the said fats, oils, waxes and other fatty substances, as well as water and an emulsifier, as usually used for a composition of this type.

The lipid phase may advantageously be selected from the following group of substances:
mineral oils, mineral waxes
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleo-gels or hydrodispersions or lipodispersions is advantageously selected from the group consisting of esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, or from the group consisting of esters of aromatic carboxylic acids and saturated and/or unsaturated, branched and/or unbranched alcohols having a chain length of 3 to 30 C atoms. Ester oils of this type can then advantageously be selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group consisting of branched and unbranched hydrocarbons and waxes, silicone oils, dialkyl ethers, or the group consisting of saturated or unsaturated, branched or unbranched alcohols, and fatty acid triglycerides, specifically the triglycerol esters of saturated and/or unsaturated, branched and/or unbranched alkanecarboxylic acids having a chain length of 8 to 24 C atoms, in particular 12-18 C atoms. The fatty acid triglycerides may advantageously be selected, for example, from the group consisting of synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as the only lipid component of the oil phase.

The oil phase is advantageously selected from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride and dicapryl ether.

Particularly advantageous are mixtures of C₁₂₋₁₅-alkyl benzoate and 2-ethylhexyl isostearate, mixtures of C₁₂₋₁₅-alkyl benzoate and isotridecyl isononanoate, as well as mixtures of C₁₂₋₁₅-alkyl benzoate, 2-ethylhexyl isostearate and isotridecyl isononanoate.

Of the hydrocarbons, paraffin oil, squalane and squalene may advantageously be used for the purposes of the present invention.

Furthermore, the oil phase may also advantageously have a content of cyclic or linear silicone oils or consist entirely of oils of this type, although it is preferred to use an additional content of other oil-phase components in addition to the silicone oil or the silicone oils.

The silicone oil to be used in accordance with the invention is advantageously cyclomethicone (octamethylcyclotetrasiloxane). However, it is also advantageous for the purposes of the present invention to use other silicone oils, for example hexamethylcyclotrisiloxane, polydimethylsiloxane, poly(methylphenylsiloxane).

Also particularly advantageous are mixtures of cyclomethicone and isotridecyl isononanoate and of cyclomethicone and 2-ethylhexyl isostearate.

The aqueous phase of the compositions according to the invention optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol, glycerol, and, in particular, one or more thickeners, which may advantageously be selected from the group consisting of silicon dioxide, aluminium silicates, polysaccharides and derivatives thereof, for example hyaluronic acid, xanthan gum, hydroxypropylmethylcellulose, particularly advantageously from the group consisting of the polyacrylates, preferably a polyacrylate from the group consisting of the so-called Carbopols, for example Carbopol grades 980, 981, 1382, 2984, 5984, in each case individually or in combination.

In particular, mixtures of the above-mentioned solvents are used. In the case of alcoholic solvents, water may be a further constituent.

Emulsions according to the invention are advantageous and comprise, for example, the said fats, oils, waxes and other fatty substances, as well as water and an emulsifier, as usually used for a formulation of this type.

In a preferred embodiment, the compositions according to the invention comprise hydrophilic surfactants.

The hydrophilic surfactants are preferably selected from the group consisting of the alkylglucosides, acyl lactylates, betaines and coconut amphoacetates.

The alkylglucosides are themselves advantageously selected from the group consisting of the alkylglucosides which are distinguished by the structural formula

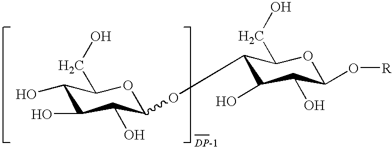

where R represents a branched or unbranched alkyl radical having from 4 to 24 carbon atoms, and where D̅P̅ denotes a mean degree of glucosylation of up to 2.

The value D̅P̅ represents the degree of glucosidation of the alkylglucosides used in accordance with the invention and is defined as

$$\overline{DP} = \frac{{p}_{1}}{100} \cdot 1 + \frac{{p}_{2}}{100} \cdot 2 + \frac{{p}_{3}}{100} \cdot 3 + … = Σ \frac{{p}_{i}}{100} \cdot i$$

in which p₁, p₂, p₃ . . . pᵢ represent the proportion of mono-, di-, tri- . . . i-fold glucosylated products in percent by weight. Products having degrees of glucosylation of 1-2, particularly advantageously of 1.1 to 1.5, very particularly advantageously of 1.2-1.4, in particular of 1.3, are advantageously selected in accordance with the invention.

The value DP takes into account the fact that alkylglucosides are generally, as a consequence of their preparation, in the form of mixtures of mono- and oligoglucosides. A relatively high content of monoglucosides, typically in the order of 40-70% by weight, is advantageous in accordance with the invention.

Alkylglucosides which are particularly advantageously used in accordance with the invention are selected from the group consisting of octyl glucopyranoside, nonyl glucopyranoside, decyl glucopyranoside, undecyl glucopyranoside, dodecyl glucopyranoside, tetradecyl glucopyranoside and hexadecyl glucopyranoside.

It is likewise advantageous to employ natural or synthetic raw materials and auxiliaries or mixtures which are distinguished by an effective content of the active ingredients used in accordance with the invention, for example Plantaren® 1200 (Henkel KGaA), Oramix® NS 10 (Seppic).

The acyllactylates are themselves advantageously selected from the group consisting of the substances which are distinguished by the structural formula

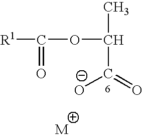

where R¹ denotes a branched or unbranched alkyl radical having 1 to 30 carbon atoms, and M⁺ is selected from the group consisting of the alkali metal ions and the group consisting of ammonium ions which are substituted by one or more alkyl and/or by one or more hydroxyalkyl radicals, or corresponds to half an equivalent of an alkaline earth metal ion.

For example, sodium isostearyl lactylate, for example the product Pathionic® ISL from the American Ingredients Company, is advantageous.

The betaines are advantageously selected from the group consisting of the substances which are distinguished by the structural formula

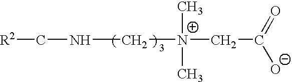

where R² denotes a branched or unbranched alkyl radical having 1 to 30 carbon atoms.

R² particularly advantageously denotes a branched or unbranched alkyl radical having 6 to 12 carbon atoms.

For example, capramidopropylbetaine, for example the product Tego® Betain 810 from Th. Goldschmidt AG, is advantageous.

A coconut amphoacetate which is advantageously selected in accordance with the invention is, for example, sodium coconut amphoacetate, as available under the name Miranol® Ultra C32 from Miranol Chemical Corp.

The compositions according to the invention are advantageously characterised in that the hydrophilic surfactant(s) is (are) present in concentrations of 0.01-20% by weight, preferably 0.05-10% by weight, particularly preferably 0.1-5% by weight, in each case based on the total weight of the composition.

For use, the cosmetic and dermatological compositions according to the invention are applied to the skin and/or the hair in an adequate amount in the usual manner for cosmetics.

Cosmetic and dermatological compositions according to the invention may exist in various forms. Thus, they may be, for example, a solution, a water-free composition, an emulsion or microemulsion of the water-in-oil (W/O) type or of the oil-in-water (O/W) type, a multiple emulsion, for example of the water-in-oil-in-water (W/O/W) type, a gel, a solid stick, an ointment or an aerosol. It is also advantageous to administer ectoines in encapsulated form, far example in collagen matrices and other conventional encapsulation materials, for example as cellulose encapsulations, in gelatine, wax matrices or liposomally encapsulated.

In particular, wax matrices, as described in DE-A 43 08 282, have proven favourable. Preference is given to emulsions. O/W emulsions are particularly preferred. Emulsions, W/O emulsions and O/W emulsions are obtainable in a conventional manner.

Emulsifiers that can be used are, for example, the known W/O and O/W emulsifiers. It is advantageous to use further conventional co-emulsifiers in the preferred O/W emulsions according to the invention.

An emulsifier that has proven to be particularly preferred in accordance with the invention for O/W emulsions is the commercial product Ceralution C from Sasol.

Co-emulsifiers which are advantageously selected in accordance with the invention are, for example, O/W emulsifiers, principally from the group consisting of the substances having HLB values of 11-16, very particularly advantageously having HLB values of 14.5-15.5, so long as the O/W emulsifiers have saturated radicals R and R′. If the O/W emulsifiers have unsaturated radicals R and/or R′ or if isoalkyl derivatives are present, the preferred HLB value of such emulsifiers may also be lower or higher.

It is advantageous to select the fatty alcohol ethoxylates from the group consisting of ethoxylated stearyl alcohols, cetyl alcohols, cetylstearyl alcohols (cetearyl alcohols). Particular preference is given to the following: polyethylene glycol (13) stearyl ether (steareth-13), polyethylene glycol (14) stearyl ether (steareth-14), polyethylene glycol (15)stearyl ether (steareth-15), polyethylene glycol (16) stearyl ether (steareth-16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20), polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12)oleyl ether (oleth-12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetylstearyl ether (ceteareth-13), polyethylene glycol (14) cetylstearyl ether (ceteareth-14), polyethylene glycol (15) cetylstearyl ether (ceteareth-15), polyethylene glycol (16) cetylstearyl ether (ceteareth-16), polyethylene glycol (17) cetylstearyl ether (ceteareth-17), polyethylene glycol (18) cetylstearyl ether (ceteareth-18), polyethylene glycol (19) cetylstearyl ether (ceteareth-19), polyethylene glycol (20) cetylstearyl ether (ceteareth-20).

It is furthermore advantageous to select the fatty acid ethoxylates from the following group:

polyethylene glycol (20) stearate, polyethylene glycol (21) stearate, polyethylene glycol (22)stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19)isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium laureth-11 carboxylate. An alkyl ether sulfate which can advantageously be used is sodium laureth-14 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group consisting of polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/caprinate, polyethylene glycol (20) glyceryl oleate, polyethylene glycol (20) glyceryl isostearate, polyethylene glycol (18) glyceryl oleate/cocoate.

It is likewise favourable to select the sorbitan esters from the group consisting of polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

The following can be employed as optional W/O emulsifiers, but ones which may nevertheless be advantageous in accordance with the invention:

fatty alcohols having 8 to 30 carbon atoms, monoglycerol esters of saturated and/or unsaturated, branched and/or unbranched alkanecarboxylic acids having a chain length of 8 to 24, in particular 12-18 C atoms, diglycerol esters of saturated and/or unsaturated, branched and/or unbranched alkanecarboxylic acids having a chain length of 8 to 24, in particular 12-18 C atoms, monoglycerol ethers of saturated and/or unsaturated, branched and/or unbranched alcohols having a chain length of 8 to 24, in particular 12-18 C atoms, diglycerol ethers of saturated and/or unsaturated, branched and/or unbranched alcohols having a chain length of 8 to 24, in particular 12-18 C atoms, propylene glycol esters of saturated and/or unsaturated, branched and/or unbranched alkanecarboxylic acids having a chain length of 8 to 24, in particular 12-18 C atoms, and sorbitan esters of saturated and/or unsaturated, branched and/or unbranched alkanecarboxylic acids having a chain length of 8 to 24, in particular 12-18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate.

Compositions which are preferred in accordance with the invention are particularly suitable for protecting human skin against UV-induced ageing processes and against oxidative stress, i.e. against damage caused by free radicals, as are generated, for example, by sunlight, heat or other influences. In this connection, they are in the various administration forms usually used for this application. For example, they may, in particular, be in the form of a lotion or emulsion, such as in the form of a cream or milk (O/W, W/O, O/W/O, W/O/W), in the form of oily-alcoholic, oily-aqueous or aqueous-alcoholic gels or solutions, in the form of solid sticks or may be formulated as an aerosol.

The composition may comprise cosmetic adjuvants that are usually used in this type of composition, such as, for example, thickeners, softeners, moisturisers, surface-active agents, emulsifiers, preservatives, antifoams, perfumes, waxes, lanolin, propellants, dyes and/or pigments which colour the composition itself or the skin, and other ingredients usually used in cosmetics.

The dispersant or solubiliser used can be an oil, wax or other fatty substance, a lower monoalcohol or a lower polyol or mixtures thereof. Particularly preferred monoalcohols or polyols include ethanol, i-propanol, propylene glycol, glycerol and sorbitol.

A preferred embodiment of the invention is an emulsion in the form of a protective cream or milk which, apart from the compounds/extracts according to the invention, comprises, for example, fatty alcohols, fatty acids, fatty acid esters, in particular triglycerides of fatty acids, lanolin, natural and synthetic oils or waxes and emulsifiers in the presence of water.

Further preferred embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

The composition according to the invention may also be in the form of an alcoholic gel which comprises one or more lower alcohols or polyols, such as ethanol, propylene glycol or glycerol, and a thickener, such as siliceous earth. The oily-alcoholic gels also comprise natural or synthetic oil or wax.

The solid sticks consist of natural or synthetic waxes and oils, fatty alcohols, fatty acids, fatty acid esters, lanolin and other fatty substances.

If a composition is formulated as an aerosol, the customary propellants, such as alkanes, fluoroalkanes and chlorofluoroalkanes, are generally used.

The cosmetic composition may also be used to protect the hair against photochemical damage in order to prevent colour changes, bleaching or damage of a mechanical nature. In this case, a suitable formulation is in the form of a rinse-out shampoo, lotion, gel or emulsion, the composition in question being applied before or after shampooing, before or after colouring or bleaching or before or after permanent waving. It is also possible to select a composition in the form of a lotion or gel for styling and treating the hair, in the form of a lotion or gel for brushing or laying a water wave, in the form of a hair lacquer, permanent-waving composition, colorant or bleach for the hair. The composition may comprise various adjuvants used in this type of composition, such as surface-active agents, thickeners, polymers, softeners, preservatives, foam stabilisers, electrolytes, organic solvents, silicone derivatives, oils, waxes, antigrease agents, dyes and/or pigments which colour the composition itself or the hair, or other ingredients usually used for hair care.

Various cosmetic products or cosmetic preparations which comprise a plant extract or a mixture thereof as described above will be described below as practical working examples of the invention.

### EXAMPLE 1

### Tiliroside-Containing Plant Extract

20 kg of Waltheria paniculata leaves are ground (mesh width 5 mm) and extracted with 250 l of ethanol for one hour with stirring. After removal of the ethanol, the operation is repeated with the same amount of ethanol.

The ethanol separated off from the residue is concentrated to about 20 l under reduced pressure, and the concentrate is cooled to 10° C. ice-water is added to the concentrate, and lipophilic constituents are separated off using 2 kg of diatomaceous earth. After filtration, the filtrate is concentrated at elevated temperature under reduced pressure, and, after cooling, a tiliroside-containing extract is obtained.

Formulations of this extract which are suitable for topical application exhibit an inflammation-inhibiting action. A 1% cream significantly reduces inflammation symptoms in neurodermatitis.

### EXAMPLE 2

A cosmetic product according to the invention in the form of a skin lightening cream can have, for example, a composition by weight as indicated below comprising the following constituent groups A, B and C.

### Constituent Group A:

| | |
|---|---|
| Glyceryl stearate (and ceteareth-20) | 15.00% |
| Paraffin oil | 3.00% |
| Ascorbyl palmitate | 3.00% |
| Dimethicone | 3.00% |
| Cetyl alcohol | 0.50% |
| PEG-30/glyceryl isostearate | 2.00% |

### Constituent Group B:

| | |
|---|---|
| Water | 72.20% |
| Methylparaben | 0.20% |
| Imidazolidinylurea | 0.30% |
| Extract according to Example 1 | 0.50% |

### Constituent Group C:

| | |
|---|---|
| Perfume | 0.30% |

The process for the preparation and production of the above-mentioned cream consists in melting the constituents of constituent group A at 75° C. with stirring, preparing constituent group B at 75° C., then pouring constituent group A into constituent group B while mixing using a turbostirrer, allowing the mixture to cool while stirring using a planetary stirrer, and adding constituent group C.

### EXAMPLE 3

A cosmetic product according to the invention in the form of an anti-blemish hand emulsion can have, for example, a composition by weight as indicated below comprising the following constituent groups A, B and C.

This emulsion may also in accordance with the invention represent a multiply active product, in particular against free radicals, UVA, UVB, proteases and glycation, having a cell metabolism-stimulating action.

### Constituent Group A:

| | |
|---|---|
| Glyceryl stearate (and PEG 100 stearate) | 6.00% |
| Oleyl alcohol | 1.50% |
| Glyceryl stearate | 2.00% |
| Steareth-2 | 2.00% |
| Shea butter | 3.00% |
| Dimethicone | 4.00% |
| Caprylic acid/capric acid triglyceride | 8.00% |
| Propylparaben | 0.10% |
| Tocopherol acetate | 0.10% |

### Constituent Group B:

| | |
|---|---|
| Water | 62.30% |
| Elestab 388 | 2.50% |
| (Laboratoires Sérobiologiques) | |
| Extract according to Example 1 | 1.00% |
| Propylene glycol | 5.00% |

### Constituent Group C:

| | |
|---|---|
| Polyacrylamide (and) isoparaffin (and) laureth-7 | 2.00% |

The process for the preparation and production of the above-mentioned emulsion consists in preparing constituent groups A and B separately at 75° C., adding constituent group A to constituent group B at 75° C. while stirring using a turbostirrer, then cooling the resultant mixture to 50° C., adding constituent group C and finally cooling the final mixture to ambient temperature.

### EXAMPLE 4

A cosmetic product according to the invention in the form of a conditioner for dry hair against stresses and for light protection can have, for example, a composition by weight as indicated below comprising the following constituent groups A and B.

### Constituent Group A:

| | |
|---|---|
| Cetyl alcohol | 2.00% |
| Paraffin oil | 2.00% |
| Sorbitol stearate | 1.00% |
| Isopropyl palmitate (and) castor oil | 1.00% |

### Constituent Group B:

| | |
|---|---|
| Glycerol | 2.00% |
| Laureth-20 | 1.00% |
| Cetrimonium chloride | 2.00% |
| Extract according to Example 1 | 0.60% |
| Elestab 388 | 1.50% |
| (Laboratoires Sérobiologiques) | |
| Water | to 100.00% |

The process for the preparation and production of the above-mentioned conditioner consists in preparing constituent groups A and B separately at 80° C. with stirring, adding fraction. A to fraction B while stirring using a turbostirrer, and finally cooling the resultant mixture to ambient temperature.

### EXAMPLE 5

A cosmetic product according to the invention in the form of a protecting body cream against stresses can have, for example, a composition by weight as indicated below comprising the following constituent groups A, B and C.

### Constituent Group A:

| | |
|---|---|
| Glyceryl stearate (and) ceteareth-20 (and) | 6.00% |
| ceteareth-10 (and) cetostearyl alcohol (and) | |
| cetyl palmitate | |
| Cetostearyl alcohol | 1.00% |
| Decyl oleate | 3.00% |
| Paraffin oil | 4.00% |
| Shea butter | 2.00% |

### Constituent Group B:

| | |
|---|---|
| Glycerol | 3.00% |
| Wheat protein hydrolysate | 0.50% |
| Extract according to Example 1 | 2.05% |
| Water | 78.25% |

### Constituent Group C:

| | |
|---|---|
| Perfume | 0.20% |

The process for the preparation and production of the above-mentioned emulsion consists in preparing constituent groups A and B separately at 80° C. with stirring, then adding fraction A to fraction B while stirring using a turbostirrer, subsequently bringing the resultant mixture to ambient temperature, and adding constituent group C.

### EXAMPLE 6

A cosmetic product according to the invention in the form of a multiply active day cream against ageing (against free radicals, UVA and elastase) can have, for example, a composition by weight as indicated below comprising the following constituent groups A, B and C.

### Constituent Group A:

| | |
|---|---|
| Glyceryl stearate | 14.00% |
| Octyldodecanol | 6.00% |
| Dibutyl adipate | 6.00% |
| Ceteareth-12 | 1.50% |
| Ceteareth-20 | 1.50% |

### Constituent Group B:

| | |
|---|---|
| Propylene glycol | 5.00% |
| Extract according to Example 1 | 2.25% |
| Elestab 4112 | 0.40% |
| (Laboratoires Sérobiologiques) | |
| Water | to 100.00% |

### Constituent Group C:

| | |
|---|---|
| Perfume | 0.20% |

The process for the preparation and production of the above-mentioned cream consists in preparing constituent groups A and B separately at 80° C. with stirring, then adding constituent group A to constituent group B while stirring using a turbostirrer, then cooling the resultant mixture to 45° C., then adding constituent group C, and finally bringing the final mixture to ambient temperature.

### EXAMPLE 7

### Constituent Group A

| | |
|---|---|
| Paraffin | 8.00% |
| Trilaureth 4-phosphate | 1.50% |
| Polyglyceryl 2-sesquiisostearate | 2.00% |
| Isopropyl palmitate | 6.00% |
| Ethylhexyl stearate | 5.00% |
| Carbomer | 0.40% |

### Constituent Group B

| | |
|---|---|
| Glycerol | 3.00% |
| Water | to 68.60% |

### Constituent Group C

| | |
|---|---|
| Extract according to Example 1 | 0.50% |
| Water | 5.00% |

### Constituent Group D

| | |
|---|---|
| Triethanolamine | q.s. |

Phase B is stirred into phase A, phase C is subsequently added, and the mixture is neutralised using phase D.

### EXAMPLE 8

### Constituent Group A

| | |
|---|---|
| Paraffin | 8.00% |
| Arlacel 481 | 6.00% |
| Isopropyl palmitate | 7.00% |
| Ethylhexyl ethylhexanoate | 4.00% |

### Constituent Group B

| | |
|---|---|
| Glycerol | 3.00% |
| Magnesium sulfate | 0.50% |
| Methylparaben, propylparaben, propylene | 0.05% |
| glycol, diazolidinylurea | |
| Water | 65.20% |

### Constituent Group C

| | |
|---|---|
| Water | 5.00% |
| Sodium hydroxide | 0.30% |
| Extract according to Example 1 | 0.50% |

The heated phase B is slowly stirred into the heated phase E. After homogenisation, phase C is added at about 30° C., and the mixture is allowed to cool with stirring.

### EXAMPLES 9 TO 16

The following table shows a number of formulation examples:

### Composition of Face Lotions Having a Lightening Action

| | Composition | | | | | | | |
|---|---|---|---|---|---|---|---|---|
| | 9 | 10 | 11 | 12 | 13 | 14 | 15 | 16 |
| Ethanol, 95% by weight | 50.0 | 50.0 | 50.0 | 50.0 | 50.0 | 50.0 | 50.0 | 50.0 |
| Propylene glycol + 8EO | 1.0 | 1.0 | 1.0 | 1.0 | 1.0 | 1.0 | 1.0 | 1.0 |
| monococoate | | | | | | | | |
| 2-Ethylhexyl salicylate | 2.0 | 2.0 | 2.0 | — | — | — | 2.0 | 2.0 |
| Tocopherol acetate | — | — | — | 1.0 | 1.0 | 1.0 | — | — |
| Thioglycerol | 0.05 | 0.05 | 0.05 | 0.05 | 0.05 | 0.05 | 0.05 | 0.05 |
| Extract of Waltheria paniculata | 1.0 | 1.0 | 1.0 | 1.0 | 1.0 | 1.0 | 1.0 | 1.0 |
| Ascorbic acid | 1.0 | — | — | 1.0 | — | — | 0.5 | — |
| Ferulic acid | — | 1.0 | — | — | 1.0 | — | 0.5 | 0.5 |
| Kojic acid | — | — | 1.0 | — | — | 1.0 | — | 0.5 |
| Sodium sulfite | 0.1 | 0.1 | 0.1 | 0.1 | 0.1 | 0.1 | 0.1 | 0.1 |
| Sodium hydrogensulfite | 0.25 | 0.25 | 0.25 | 0.25 | 0.25 | 0.25 | 0.25 | 0.25 |
| Aluminium nitrate | 0.5 | 0.5 | 0.5 | 0.5 | 0.5 | 0.5 | 0.5 | 0.5 |
| Perfume oil | 0.05 | 0.05 | 0.05 | 0.05 | 0.05 | 0.05 | 0.05 | 0.05 |

| | |
|---|---|
| Water | to 100 |

The invention is of course not restricted to the embodiments described above. Modifications, in particular in relation to the composition of individual elements or through the use of corresponding other techniques, are possible without going beyond the scope of the invention.

### EXAMPLE 17

### Isolation of Tiliroside from Waltheria paniculata

20 kg of Waltheria paniculata leaves are ground (mesh width 5 mm) and extracted with 250 l of ethanol for one hour with stirring. After removal of the ethanol, the operation is repeated with the same amount of ethanol.

The ethanol separated off from the residue is concentrated to about 20 l under reduced pressure, and the concentrate is cooled to 10° C. Ice-water is added to the concentrate, and lipophilic constituents are separated off using 2 kg of diatomaceous earth. After filtration, the filtrate is again concentrated at elevated temperature under reduced pressure, and, after cooling, crude tiliroside is obtained as filter cake.

The filter cake is suspended in ethanol and heated, water is added, and the mixture is stirred under reflux. The product is filtered off, washed with ethanol and dried.

The tiliroside obtained can be employed in cosmetic formulations in a manner known per se.

1-17. (canceled)

18. A process for preparing tiliroside from plant material, comprising extracting parts of a plant, wherein the parts of a plant are from a plant that is Waltheria douradinha, Waltheria paniculata, Waltheria indica, Waltheria viscosissima, Waltheria antennalis, Waltheria ovata, Waltheria tomentosa, Waltheria madagascariensis, Waltheria glomerata, Waltheria bicolor, Waltheria fryxellii, Waltheria lundelliana, Waltheria tridentata, Waltheria operculata, Waltheria bracteosa, Waltheria macropoda, Waltheria caroliniana, Waltheria arenicola, Waltheria melochia, Waltheria acuminata, Waltheria theobroma, Waltheria indivia or Waltheria taiwana, and the resultant extract is processed further.

19. The process according to claim 18, wherein the plant is Waltheria paniculata.

20. The process according to claim 18, wherein the parts of the plant are leaves.

21. The process according to claim 18, wherein the resultant extract is processed further by purifying tiliroside.

22. The process according to claim 18, wherein the resultant extract is processed further by purifying tiliroside by recrystallisation or washing.

23. A process for preparing tiliroside from plant material, comprising extracting parts of a plant, wherein the parts of a plant are from a plant that is Waltheria americana, Waltheria douradinha, Waltheria paniculata, Waltheria indica, Waltheria viscosissima, Waltheria antennalis, Waltheria ovata, Waltheria tomentosa, Waltheria madagascariensis, Waltheria glomerata, Waltheria bicolor, Waltheria Waltheria lundelliana, Waltheria tridentata, Waltheria operculata, Waltheria bracteosa, Waltheria macropoda, Waltheria caroliniana, Waltheria arenicola, Waltheria melochia, Waltheria acuminata, Waltheria theobroma, Waltheria indivia or Waltheria taiwana, wherein the parts of the plant are
a. comminuted in a first step and
b. extracted with a polar organic solvent in a second step,
c. water is added to the solution in a third step,
d. the solution is concentrated in a fourth step, and
e. resultant tiliroside is precipitated by cooling in a fifth step.

24. The process according to claim 23, wherein in the third step, stirring is carried out at a temperature in the range 0° C.-25° C., and any solids precipitating are filtered off.

25. The process according to claim 23, wherein in the second step, the extraction is carried out under reflux.

26. The process according to claim 23, wherein the resultant tiliroside is purified in a sixth step.

27. The process according to claim 23, wherein the plant parts are extracted with a polar organic solvent at a temperature of 85-90° C. in a second step.

28. The process according to claim 23, wherein in the second step, the extraction is carried out under reflux, and methanol or ethanol is employed as solvent.

29. The process according to claim 23, wherein the resultant tiliroside is purified in a sixth step by recrystallisation or washing.

31. The Process according to claim 23, wherein the plant is Waltheria paniculata.

32. The process according to claim 23, wherein the plant parts are leaves.

33. A process for preparing tiliroside from plant material, comprising extracting parts of a plant, wherein the parts of a plant are from Waltheria paniculata,
wherein the parts of the plant are
a. comminuted in a first step and
b. extracted with a polar organic solvent in a second step,
c. water is added to the solution in a third step,
d. the solution is concentrated in a fourth step, and
e. resultant tiliroside is precipitated by cooling in a fifth step.
f. purifying the resultant tiliroside.
